(19) United States
(12) Patent Application Publication
(10) Pub. No.: US 2010/0021901 A1
(43) Pub. Date: Jan. 28, 2010

(54) Compositions and methods for detecting analytes

(76) Inventors: Peng Yin, Pasadena, CA (US); Niles A. Pierce, Pasadena, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/454,799

(22) Filed: May 22, 2009

Related U.S. Application Data
(60) Provisional application No. 61/128,551, filed on May 22, 2008.

(51) Int. Cl. C12Q 1/68; C07H 21/04

(52) U.S. Cl. 435/6; 536/24.3

(57) ABSTRACT

Embodiments disclosed herein relate generally to probes (e.g. self-quenching probes), methods, and kits for detecting the presence of a target analyte using probes.

### CROSS-REFERENCE TO RELATED APPLICATIONS

The present application claims priority to U.S. Provisional Application No. 61/128,551, filed May 22, 2008, which is hereby incorporated by reference in its entirety.

### SEQUENCE LISTING

The present application is being filed along with a Sequence Listing in electronic format. The Sequence Listing is provided as a file entitled CALTE.048A.txt, created May 22, 2009, which is 577 bytes in size. The information in the electronic format of the Sequence Listing is incorporated herein by reference in its entirety.

### STATEMENT REGARDING FEDERALLY SPONSORED R&D

This invention was made with government support under grant nos. NIH 5R01EB006192-04 “Hybridization chain reaction: in situ amplification for biological imaging” and NIH P50 HG004071 “Center for in toto genomic analysis of vertebrate development”.

### BACKGROUND OF THE INVENTION

1. Field of the Invention

The present application relates generally to compositions and methods for detecting the presence of a target analyte.

2. Description of the Related Art

Molecular beacons are being used as research tools for bioimaging. A molecular beacon is a single-stranded nucleic acid hybridization probe that forms a stem-and-loop structure. The loop is complementary to the target nucleic acid sequence and can hybridize to the target in its presence. A fluorophore and a quencher are attached to the ends of the stem such that the fluorophore is quenched by the quencher in the stem-loop configuration. Detection of the target brings the beacon to a hybrid configuration. The resultant spatial separation of the fluorophore from the quencher enables the fluorophore to fluoresce brightly (S. Tyagi et al. 1996 Nat Biotechnol 14:303-308; D. P. Bratu 2006 Methods Mol Biol 319:1-14). The fact that non-specific binding to the molecular beacon can trigger a conformational change and generate signal and that only one fluorophore is attached to a beacon are key limitations of the molecular beacon technology.

### SUMMARY OF THE INVENTION

In some embodiments, probes for detecting the presence of a target nucleic acid analyte in a sample (e.g., cell) are provided. In some embodiments, the probes generally comprise a first nucleic acid strand; a second nucleic acid strand hybridized to the first nucleic acid strand; a duplex region formed between the first nucleic acid strand and the second nucleic acid strand, wherein the duplex region comprises one or more moiety pairs, wherein the moiety pair comprises a first moiety attached to a first nucleotide of the first nucleic strand and a second moiety attached to a second nucleotide of the second nucleic strand, wherein a first signal can be detected from one or more moieties of the moiety pair when the first nucleic acid strand is hybridized to the second nucleic acid strand, wherein the first signal is different than a second signal that can be detected from one or more moieties of the moiety pair when the first nucleic acid strand is not hybridized to the second nucleic acid strand; and a first toe-hold region comprising a first single-stranded region of the first nucleic acid strand that extends beyond the second nucleic acid strand, wherein in the presence of the target nucleic acid analyte, the first nucleic strand and the second nucleic acid strand separate, such that the second signal can be detected.

In some embodiments, the probe for detecting the presence of a target analyte in a sample comprises a first nucleic acid strand; a second nucleic acid strand comprising a first moiety, wherein the first nucleic acid strand and the second nucleic are hybridized to each other; a third nucleic acid strand comprising a second moiety, wherein the first nucleic acid strand and the third nucleic are hybridized to each other; wherein said first moiety is in proximity (adjacent) to said second moiety such that a first signal that can be detected from one or more moieties of the moiety pair that is different than when a second signal that can be detected from one or more moieties of the moiety pair when the said first moiety is not in proximity (adjacent) to said second moiety (e.g., upon strand displacement).

In some embodiments, a portion of the first toe-hold region is complementary to a portion of the target nucleic acid analyte. In some embodiments, a portion of the first toe-hold region is complementary to a first portion of a first monomer, wherein a second portion of said first monomer is complementary to a first portion of a second monomer, and wherein a second portion of said second monomer is complementary to a portion of the target nucleic acid analyte. In some embodiments, the first toe-hold region comprises a length of about 4 to about 50 nucleotides.

In some embodiments, the probe further comprises a second toe-hold region comprising a second region of the first nucleic acid strand that extends beyond the second nucleic acid strand.

In some embodiments, the duplex region comprises a length of about 8 to about 50 nucleotides. In some embodiments, said duplex region comprises one moiety pair. In some embodiments, said moiety pair is located in a portion of the probe that is at the opposite end from said toehold region. In some embodiments, the first moiety comprises a fluorophore and the second moiety comprises one or more quenchers. In some embodiments, the first moiety comprises a fluorophore and the second moiety comprises a fluorophore.

In some embodiments, the duplex region comprises multiple moiety pairs. In some embodiments, each moiety pair comprises a fluorophore and a quencher. In some embodiments, said fluorophores are spectrally distinct. In some embodiments, said fluorophores are spectrally indistinct. In some embodiments, each base pair in the duplex region comprises a moiety pair.

Some embodiments provide for a kit comprising a probe described herein and instructions for use.

In some embodiments, methods for detecting the presence of a target analyte (e.g., a nucleic acid) in a sample are provided. In some embodiments, the methods generally comprise contacting the sample with a probe, wherein the probe comprises: a first nucleic acid strand; a second nucleic acid strand hybridized to the first nucleic acid strand; one or more moiety pairs, wherein the moiety pair comprises a first moiety attached to the first nucleic acid strand and a second moiety attached to the second nucleic acid strand, wherein a first signal can be detected from one or more moieties of the moiety pair when the first nucleic acid strand is hybridized to the second nucleic acid strand; and a first toe-hold region comprising a first single-stranded region of the first nucleic acid strand that extends beyond the second nucleic acid strand, wherein a portion of the first toe-hold region is substantially complementary to a portion of the target or other molecule (e.g., a nucleic acid hairpin monomer); wherein in the presence of said target analyte or said other molecule in the sample, the first nucleic acid strand is displaced from said second nucleic acid strand and a second signal is generated that can be detected from one or more moieties of the moiety pair; and measuring the first signal and the second signal, detecting the presence of the target analyte when the second signal is different than the first signal.

In some embodiments, the method further comprises contacting the sample with a first monomer and a second monomer wherein the target analyte comprises a first monomer binding region that is complementary to a first portion of said first monomer wherein the first monomer comprises a second monomer binding region that is complementary to a first portion of said second monomer wherein said second monomer comprises a first toe-hold binding region that is complementary to a portion of the first toe-hold region; wherein binding of the target analyte to the first toe-hold region initiates the displacement of said first nucleic acid strand from said second nucleic acid strand and generates the second signal. In some embodiments, the first and second monomers comprise RNA hairpin monomers with sticky ends.

In some embodiments, the first toe-hold region comprises a length of about 4 to about 50 nucleotides.

In some embodiments, the methods further comprise a second toe-hold region comprising a second single-stranded region of the first nucleic acid strand that extends beyond the second nucleic acid strand, wherein a portion of the second toe-hold region is substantially complementary to the analyte.

In some embodiments, said moiety pair is located in a portion of the probe that is at the opposite end from said toehold region. In some embodiments, the first moiety comprises a fluorophore and the second moiety comprises one or more quenchers. In some embodiments, the first moiety comprises a fluorophore and the second moiety comprises a fluorophore.

In some embodiments, the probe comprises multiple moiety pairs. In some embodiments, each moiety pair comprises a fluorophore and a quencher. In some embodiments, said fluorophores are spectrally distinct. In some embodiments, said fluorophores are spectrally indistinct. In some embodiments, the target analyte is associated with a disease or disorder. In some embodiments, the target analyte is an mRNA molecule.

### BRIEF DESCRIPTION OF THE DRAWINGS

FIG. 1 schematically illustrates one embodiment of a probe and a method for detecting a target analyte (e.g., mRNA target). The probe comprises two single stranded nucleic acid strands, a duplex region, a moiety pair, and a toe-hold region. In the absence of the mRNA target, the probe is in a closed conformation and is self-quenching. When the mRNA target is present, the strands of the probe are displaced. This toe-hold mediated displacement reaction causes the moieties to become spatially separated, leading to a detectable change in signal from at least one moiety in the moiety pair. Circles represent quenchers and stars represent fluorophores in the figures.

FIG. 2 schematically illustrates another embodiment of a probe and a method for detecting a target analyte (e.g., mRNA target). The probe comprises two single stranded nucleic acid strands, a duplex region, multiple moiety pairs, and two toe-hold regions. In the depicted embodiment, the nucleic acid strand containing the fluorescent moieties contains multiple fluorophores that are spectrally indistinct. In the absence of the mRNA target, the probe is in a closed conformation and is self-quenching. When the mRNA target is present, the strands of the probe are displaced. This toe-hold mediated displacement reaction causes the moieties of each moiety pair to become spatially separated, leading to a detectable change in signal from at least one of the moiety pairs.

FIG. 3 schematically illustrates another embodiment of a probe and a method for detecting a target analyte (e.g., mRNA target). The probe comprises two single stranded nucleic acid strands, a duplex region, multiple moiety pairs, and two toe-hold regions. In the depicted embodiment, the nucleic acid strand containing the fluorescent moieties contains combinations of spectrally distinct fluorophores. In the absence of the mRNA target, the probe is in a closed conformation and is self-quenching. When the mRNA target is present, the strands of the probe are displaced. This toe-hold mediated displacement reaction causes the moieties of each moiety pair to become spatially separated, leading to a detectable change in signal from at least one of the moiety pairs.

FIG. 4 schematically illustrates another embodiment of a probe and a method for detecting a target analyte (e.g., mRNA target). The probe comprises two single stranded nucleic acid strands, a duplex region, multiple moiety pairs, and two toe-hold regions. In the depicted embodiment, the nucleic acid strand containing the quencher moieties contains multiple quencher moieties at each base where a moiety is present. In the absence of the mRNA target, the probe is in a closed conformation and is self-quenching. When the mRNA target is present, the strands of the probe are displaced. This toe-hold mediated displacement reaction causes the fluorescent and quencher moieties to become spatially separated, leading to a detectable change in signal from at least one of the moiety pairs.

FIG. 5 schematically illustrates another embodiment of a probe and a method for detecting a target analyte (e.g., mRNA target). The probe comprises three types of single stranded nucleic acid strands (an A strand, one or more B strands (e.g., B1, B2, and B3 in FIG. 5), and one or more C strands (e.g., C1, C2, and C3 in FIG. 5)). In the depicted embodiment, each B strand contains a quencher moiety at one end of the strand and each C strand contains one fluorescent moiety at one end of the strand, such that the C1 moiety can be quenched by the B1 moiety, the C2 moiety can be quenched by the B2 moiety, and the C3 moiety can be quenched by the B3 moiety. In the absence of the mRNA target, the B strands and the C strands are hybridized to the A strand, such that the C1 moiety is quenched by the B1 moiety, the C2 moiety is quenched by the B2 moiety, and the C3 moiety is quenched by the B3 moiety. When the mRNA target is present, the strands of the probe are displaced, and the moieties of each moiety pair become spatially separated, leading to a detectable change in signal from at least one moiety.

FIG. 6 schematically illustrates another embodiment of a probe and a method for detecting a target analyte (e.g., mRNA target). In the depicted embodiment, a probe and two monomers are shown. In the absence of the nucleic acid target T, the probe comprising strands C and D, monomer A, and monomer B co-exist metastably and do not induce strand displacement of the probe on their own. When T is present in the system, it activates A, and the complex T.A is formed. T.A, in turn, interacts with B to form T.A.B, which in turn, interacts with the probe to displace the strands of the probe and to form T.A.B.C. The moieties of each moiety pair become spatially separated, leading to a detectable change in signal.

FIG. 7 illustrates schematically illustrates another embodiment of a probe and a method for detecting a target analyte (e.g., mRNA target) using a dendritic amplification process.

FIG. 8 illustrates the generation of a self-quenched probe and the results of using the quenched probe system for in situ hybridization conditions as measured by gel electrophoresis.

FIGS. 9A and 9B demonstrate in situ verification of active background suppression with the self-quenching probe of FIG. 8.

### DETAILED DESCRIPTION

Embodiments disclosed herein relate to various probes and methods for detecting analytes using the probes described herein. In some embodiments, the probes provide for the ability to detect analytes with a higher degree of specificity than conventional probes, thereby reducing erroneous background signal produced by the probe in the absence of the analyte. In some embodiments, enhanced specificity is provided by using probes comprising two or more single-stranded nucleic acid strands and one or more single-stranded toe-hold regions. In various embodiments, the self-quenching probes and methods described herein reduce the need for additional washing steps that are otherwise typically required for methods for detecting analytes using conventional probes. As a result, the self-quenching probes described herein can be particularly useful in, for example, in vivo detection methods where washing steps are not practical.

In some embodiments, the self-quenching probes described herein utilize multiple moiety pairs (e.g., fluorescent and quencher moiety pairs). Probes containing multiple moiety pairs can provide quantitative amplification of target signal. In some embodiments, probes containing multiple moiety pairs can be used to amplify the signal obtained when target analytes present in low quantities in cells or samples are detected. In other embodiments, probes containing multiple moiety pairs can provide unique signatures or barcode-type readouts for targets by using, for example, moieties that are spectrally distinct. This allows for the detection and identification of multiple different targets in a sample.

In further embodiments, self-quenching probes described herein comprising two or more single-stranded nucleic acids can provide higher quenching efficiencies (e.g., when the moiety pair is a fluorophore and a quencher) when the probe is in a closed conformation in the absence of the target analyte compared to traditional probes.

In additional embodiments, methods for detecting analytes described herein can use probes that are not complementary to the target analyte to be detected. In such methods, one or more monomers (e.g., nucleic acid hairpin monomers) can further be used to detect the analyte of interest. Such methods permit the nucleic acid strands of the self-quenched probe to detect different target analytes. In other embodiments, the use of monomers in methods described herein provides for dendritic amplification of the signal.

The following section outlines the definitions of some of the terms used herein as well as providing some alternative embodiments. Following that section, a general description of self-quenching probes and methods for detecting analytes using such probes is provided, including various alternative embodiments. Following that section, a series of Examples outlining some possible uses of some of the disclosed embodiments is provided.

### DEFINITIONS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques of described herein are those well known and commonly used in the art.

As utilized in accordance with the embodiments provided herein, the following terms, unless otherwise indicated, shall be understood to have the following meanings:

The term “nucleic acid” refers to natural nucleic acids, artificial nucleic acids, analogs thereof, or combinations thereof. Nucleic acids may also include analogs of DNA or RNA having modifications to either the bases or the backbone. For example, nucleic acid, as used herein, includes the use of peptide nucleic acids (PNA). The term “nucleic acids” also includes chimeric molecules. Nucleic acids include, but are not limited to, DNA, cDNA, genomic DNA, mitochondrial DNA, RNA, mRNA, miRNA, siRNA, piwi-interacting RNA, rRNA, tRNA, snRNA, and viral RNA.

As used herein, the terms “polynucleotide,” “oligonucleotide,” and “nucleic acid oligomers” are used interchangeably and mean single-stranded and double-stranded polymers of nucleic acids, including, but not limited to, 2′-deoxyribonucleotides (nucleic acid) and ribonucleotides (RNA) linked by internucleotide phosphodiester bond linkages, e.g. 3′-5′ and 2′-5′, inverted linkages, e.g. 3′-3′ and 5′-5′, branched structures, or analog nucleic acids. Polynucleotides have associated counter ions, such as H⁺, NH₄⁺, trialkylammonium, Mg²⁺, Na⁺ and the like. A polynucleotide can be composed entirely of deoxyribonucleotides, entirely of ribonucleotides, or chimeric mixtures thereof. Polynucleotides can be comprised of nucleobase and sugar analogs. Polynucleotides typically range in size from a few monomeric units, e.g. 5-40 when they are more commonly frequently referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytidine, “G” denotes deoxyguanosine, and “T” denotes thymidine.

A “gene” (e.g., a marker gene) or “coding sequence” or a sequence, which “encodes” a particular protein, is a nucleic acid molecule which is transcribed (in the case of DNA) and translated (in the case of mRNA) into a polypeptide in vitro or in vivo when placed under the control of appropriate regulatory or control sequences. The boundaries of the gene are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the gene sequence.

As used herein, the terms “complementary” or “complementarity” are used in reference to polynucleotides (e.g., a sequence of nucleotides) related by the base-pairing rules. For example, the sequence “5′-A-G-T-3” is complementary to the sequence “3′-T-C-A-5′.” The nucleic acid sequences can comprise natural nucleotides (including their hydrogen bonding bases A, C, G, T, or U) and/or modified nucleotides or bases. Complementarity may be “partial,” in which less than all of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. As used herein, a hybridizing nucleic acid sequence is “substantially complementary” when it is at least 80%, more preferably at least 85%, 90%, 91%, 92%, 93%, 94%, 95%, 96%, 97%, 98%, 99% or 100%, identical and/or includes no more than one non-Watson-Crick base pairing interaction to a reference sequence in the hybridizing portion of the sequences.

The terms “hybridize,” “hybridization,” and their cognates are used herein to refer to the pairing of complementary nucleic acids or bases. Hybridization and the strength of hybridization (e.g., the strength of the association between the nucleic acids) is influenced by such factors as the degree of complementarity between the nucleic acids, stringency of the hybridization conditions involved, the melting temperature (Tm) of the formed hybrid, and the G:C ratio within the nucleic acids. A hybridizing sequence is typically at least 75%, 80%, 85%, 90%, 91%, 92%, 93%, 94%, 95%, 96%, 97%, 98%, 99% or 100% identical and is matched according to the base pairing rules. It may contain natural and/or modified nucleotides and bases.

A “cell” or “target cell” refers to a cell that contains or may contain an analyte for detection. Examples of target cells include, for example without limitation, cells that contain a nucleic acid signature for a disease, such as, for example, mutant mRNA or fusion mRNA entities. Other examples include, but are not limited to, cells that contain higher-than-background levels of mRNA, peptides, polypeptides, antibodies or fragments thereof, signal cascade molecules, viral particles, bacteria and parasitic organisms.

### Probes

Embodiments disclosed herein relate to compositions and methods that include a “self-quenching probe” or “probe” comprising two or more nucleic acid strands (e.g., a first nucleic acid strand and a second nucleic acid strand). In some embodiments, the nucleic acid strands are single-stranded.

In some embodiments, the probe comprises two nucleic acid strands such that the two nucleic strands comprise a duplex region. A duplex region refers to a region of complementarity between the two nucleic acid strands such that the two nucleic acid strands can hybridize over the length of the duplex region. In some embodiments, the length of the duplex region can be from about 2 to about 1000 bases, preferably from about 2 to about 500 bases, more preferably from about 4 to about 100 bases, or more preferably from about 5 to about 50 bases. In some embodiments, the length of the duplex region can be any number of bases between about 2 and about 1000 bases. In some embodiments, the length of the duplex region can be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases. The optimum length of the duplex region can be designed by one of ordinary skill in the art, for example, to determine the desired strand displacement conditions.

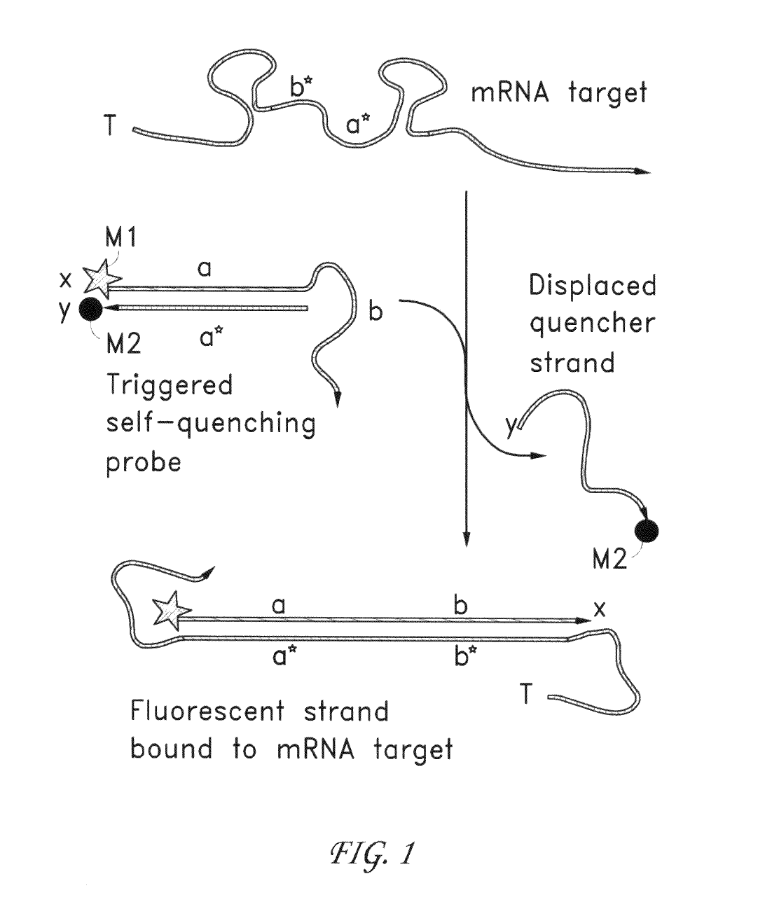

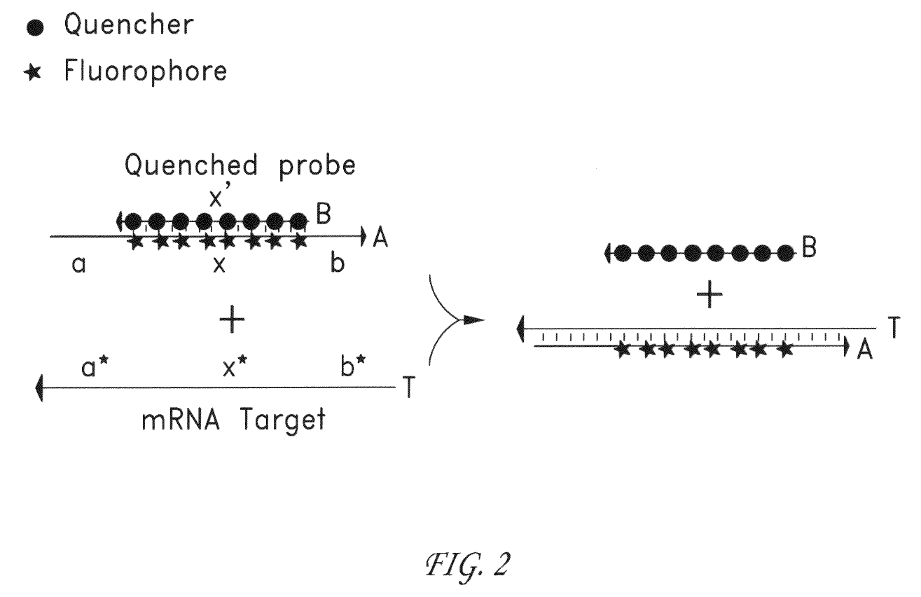

In some embodiments, one nucleic acid strand of the probe can extend beyond the other nucleic acid strand, forming one or more single-stranded toe-hold regions adjacent to the duplex region. In some embodiments, one toe-hold region is formed (see, for example, FIG. 1). In some embodiments the toe-hold region is located at the 5′ end of a nucleic acid strand. In other embodiments, the toe-hold region is located at the 3′ end of a nucleic acid strand. In some embodiments, two toe-hold regions are formed, one on either side of the duplex region (see, for example, FIG. 2). For example, a toe-hold region can be formed on the 5′ end and the 3′ end of one strand of the probe. In some embodiments, the two nucleic acid strands of the probe can be offset such that a toe-hold region is formed on each of the two nucleic acid strand (e.g., a toehold region is formed on the 5′ end of each strand or a toe-hold region is formed on the 3′ end of each strand). In some embodiments, the toe-hold region is single-stranded. In some embodiments, the toe-hold region comprises a region that is complementary to a region of the target analyte. In some embodiments, the toe-hold region does not comprise a region that is complementary to a region of the target analyte. For example, in some embodiments, the toe-hold region comprises a region that is complementary to a region of a nucleic acid monomer. In some embodiments, the length of the toe-hold region can be from about 4 to about 1000 bases, preferably from about 5 to about 500 bases, more preferably from about 6 to about 100 bases, or more preferably from about 8 to about 50 bases. In some embodiments, the length of the toe-hold region can be any number of bases between about 2 and about 1000 bases. In some embodiments, the length of the toe-hold region can be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases. The optimum length of the toe-hold region can be designed by one of ordinary skill in the art, for example, to determine the desired strand displacement conditions.

A vast variety of modified nucleic acid analogs can also be used, including backbone modifications, sugar modifications, nitrogenous base modifications, or combinations thereof. The “backbone” of a natural nucleic acid is made up of one or more sugar-phosphodiester linkages. The backbone of a nucleic acid can also be made up of a variety of other linkages known in the art, including peptide bonds, also known as a peptide nucleic acid (Hyldig-Nielsen et al., PCT No. WO 95/32305; Egholm (1992) J. Am. Chem. Soc. 114:1895; Meier et al. (1992) Chem. Int. Ed. Engl. 31:1008; Nielsen (1993) Nature 365:566; Carlsson et al. (1996) Nature 380:207); phosphorothioate linkages (Mag et al. (1991) Nucleic Acids Res. 19:1437; U.S. Pat. Nos. 5,644,048; 5,539,082; 5,773,571; 5,977,296, and 6,962,906); phosphorodithioate linkages (Briu et al. (1989) J. Am. Chem. Soc. 111:2321); phosphoramidate linkages (Beaucage et al. (1993) Tetrahedron 49(10):1925; Letsinger (1970) J. Org. Chem. 35:3800; Sprinzl et al. (1977) Eur. J. Biochem. 81:579; Letsinger et al. (1986) Nucleic Acids Res. 14:3487; Sawai et al. (1984) Chem. Lett. 805; Letsinger et al. (1988) J. Am. Chem. Soc. 110:4470; and Pauwels et al. (1986) Chemica Scripta 26:1419); methylphosphonate linkages; O-methylphosphoroamidite linkages (Eckstein, Oligonucleotides and Analogues: A Practical Approach, Oxford University Press); or combinations thereof.

Other suitable linkages include positive backbones (Denpcy et al. (1995) Proc. Natl. Acad. Sci. USA 92:6097); non-ionic backbones (U.S. Pat. Nos. 5,386,023, 5,637,684, 5,602,240, 5,216,141 and 4,469,863; Kiedrowski et al. (1991) Angew. Chem. Intl. Ed. English 30:423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research,” Ed. Y. S. Sanghui and P. Dan Cook; Mesmaeker et al. (1994), Bioorganic & Medicinal Chem. Lett. 4:395; Jeffs et al. (1994) J. Biomolecular NMR 34:17; Horn et al. (1996) Tetrahedron Lett. 37:743); and non-ribose backbones (U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook).

Sugar moieties of a nucleic acid can be either ribose, deoxyribose, or similar compounds having known substitutions, such as 2′-O-methyl ribose, 2′-halide ribose substitutions (e.g., 2′-F), and carbocyclic sugars (Jenkins et al. (1995), Chem. Soc. Rev. pp 169-176). The nitrogenous bases are conventional bases (A, G, C, T, U), known analogs thereof, such as inosine (I) (The Biochemistry of the Nucleic Acids 5-36, Adams et al., ed., 11th, 1992), known derivatives of purine or pyrimidine bases, such as N⁴-methyl deoxygaunosine, deaza- or aza-purines, deaza- or aza-pyrimidines, pyrimidine bases having substituent groups at the 5 or 6 position, purine bases having an altered or a replacement substituent at the 2, 6 or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines (Cook, PCT No. WO 93/13121) and “abasic” residues where the backbone includes no nitrogenous base for one or more residues of the polymer (Arnold et al., U.S. Pat. No. 5,585,481).

### Analytes

Some embodiments relate to methods for detecting an analyte or a plurality of analytes. The terms “analyte,” “target,” “target analyte,” and “detection target” referred to herein can be used interchangeably. In some embodiments, methods can be used to analyze biological analytes. In some embodiments, methods can be used to analyze non-biological analytes. Suitable biological analytes include, but are not limited to, nucleic acids, proteins, polypeptides, peptides, peptide nucleic acids, antibodies, antigens, receptors, molecules (e.g., a signal cascade molecule), hormones, biological cells, microorganisms (e.g., bacteria), parasitic organisms, cellular organelles, cell membrane fragments, bacteriophage, bacteriophage fragments, whole viruses, viral particles, viral fragments, and small molecules such as lipids, carbohydrates, amino acids, drug substances, and molecules for biological screening and testing. An analyte can also refer to a fused entity or a complex of two or more molecules, for example, a ribosome with both RNA and protein elements or an enzyme with substrate attached.

In some embodiments, the analyte is a nucleic acid molecule, such as DNA, cDNA, genomic DNA, mitochondrial DNA, RNA, mRNA, miRNA, siRNA, piwi-interacting RNA, rRNA, tRNA, snRNA, viral RNA, and fragments and segments thereof. An analyte or target sequence can be single-stranded, double-stranded, continuous, or fragmented, so long as a probe can be used to detect the target or a subsequence of the target. The target may be a gene, a gene fragment, or an extra-chromosomal nucleic acid sequence, for example. As used herein, a “subsequence” refers to a portion of a sequence, such as a target nucleic acid sequence, that is contained within the longer sequence.

In some embodiments, the analyte can be at least a portion of the sequence of any gene for which detection is desirable. In some embodiments, the detection target can be an mRNA. In other embodiments, the analyte can be DNA. In some embodiments, the analyte can be a portion of a nucleic acid (e.g., an mRNA) associated with a disease or disorder. In some embodiments, the analyte can be a portion of a nucleic acid not associated with a disease or disorder. In some embodiments, the analyte can be a non-biological analyte. Non-biological analytes include, but are not limited to, organic compositions, inorganic compositions and other compositions not typically found in a biological system.

Samples to be tested for analytes or sources of analytes (such as cells) can be isolated from organisms and pathogens such as viruses and bacteria or from an individual or individuals, including, but not limited to, skin, plasma, serum, spinal fluid, lymph fluid, synovial fluid, urine, tears, blood cells, organs, tumors, and also samples of in vitro cell culture constituents, such as conditioned medium resulting from the growth of cells in cell culture medium, recombinant cells and cell components. The presence of analytes can also be tested from environmental samples such as air or water samples, or from forensic samples from biological or non-biological samples, including clothing, tools, publications, letters, furniture, etc. Additionally, the presence of analytes can also be tested from synthetic sources. The probes and methods provided herein can be used in a variety of applications, such as commercial applications. For example, probes and methods described herein can be used at one or more steps in a production process to test either for one or more contaminants and/or to test for one or more desired components. The analytes can be provided in a sample that can be a crude sample, a partially purified or substantially purified sample, or a treated sample, where the sample can contain, for example, other natural components of biological samples, such as proteins, lipids, salts, nucleic acids, and carbohydrates. The presence of analytes can be tested, for example, in vitro, in situ, or ex vivo. In some embodiments, the probes described herein can be used on a chip such that binding of the target to the probe displaces the strands (e.g., quencher strand and fluorescent strand) to generate a fluorescent signal at that location on the chip. Such methods proved the ability to have tunable control over specificity relative to existing DNA chip methods.

In some embodiments, the presence of an analyte can be tested in vivo in a subject.

In some embodiments, the target analyte is preferably a nucleic acid molecule (e.g., an mRNA). In some embodiments, the nucleic acid analyte comprises a sequence that is complementary to at least a portion of the toehold region of a probe that is available for hybridization with the analyte while the probe is in a kinetically stable state. The analyte also preferably comprises a sequence that is complementary to a portion of the duplex region adjacent to the toehold region such that hybridization of the analyte to the toehold region causes a conformational change in the duplex region and initiates strand displacement of the duplex region. For example, the analyte may comprise a region that is complementary to the toe-hold region of the probe, as described above and illustrated in FIG. 1.

In some embodiments, the binding region (e.g., analyte binding region or monomer binding region) of the toe-hold region of the probe is preferably at least 80%, more preferably at least 85%, 90%, 91%, 92%, 93%, 94%, 95%, 96%, 97%, 98%, 99% or 100%, identical to at least a portion of the analyte. In preferred embodiments, the analyte binding region is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases in length.

In some embodiments, the analyte or another molecule (e.g., a monomer) is able to specifically bind to at least a portion of the probe (e.g., a portion of the toe-hold region of the probe). In some embodiments, a portion of the toe-hold region of the probe is sufficiently complementary to a portion of the target sequence or other molecule to hybridize under the selected reaction conditions. High stringency conditions are known in the art (see, for example, Maniatis et al., Molecular Cloning: A Laboratory Manual 2d Edition, 1989, and Short Protocols in Molecular Biology, ed. Ausubel, et al., both of which are hereby incorporated by reference in their entireties). Stringent conditions are typically sequence-dependent and can be different in different circumstances. Longer sequences typically hybridize specifically at higher temperatures. An extensive guide to the hybridization of nucleic acids is found, for example, in Tijssen, Techniques in Biochemistry and Molecular Biology-Hybridization with Nucleic Acid Probes, “Overview of principles of hybridization and the strategy of nucleic acid assays” (1993). Typically, stringent conditions can be selected to be about 5-10° C. lower than the thermal melting point (Tₘ) for the specific sequence at a defined ionic strength pH. The Tₘ is the temperature (under defined ionic strength, pH and nucleic acid concentration) at which 50% of the probes complementary to the target hybridize to the target sequence at equilibrium (as the target sequences are present in excess, at Tₘ 50% of the probes are occupied at equilibrium). Stringent conditions are typically those in which the salt concentration is less than about 1.0 M sodium ion concentration, typically about 0.01 to 1.0 M sodium ion concentration (or other salts) at pH of about 7.0 to about 8.3 and the temperature is at least about 30° C. for short complementary sequences (e.g. 10 to 50 nucleotides) and at least about 60° C. for long complementary sequences (e.g. greater than 50 nucleotides). Stringent conditions can also be achieved with the addition of destabilizing agents such as formamide. In other embodiments, less stringent hybridization conditions can be used. For example, moderate or low stringency conditions may be used, as are known in the art (see, for example Maniatis and Ausubel, supra, and Tijssen, supra). Any two complementary or substantially sequences described herein can be hybridized according to the conditions described above.

A probe can be referred to as “activated” or an “activated probe” when the analyte or other molecule (e.g., a monomer) is bound to or hybridized to the probe (e.g., portion of the toe-hold region of the probe). The phrase “specifically bind(s)” or “bind(s) specifically” when referring to a detection probe refers to a detection probe that has intermediate or high binding affinity, exclusively or predominately, to a target molecule. The phrase “specifically binds to” refers to a binding reaction which is determinative of the presence of a target in the presence of a heterogeneous population of other biologics. Thus, under designated assay conditions, the specified binding region binds preferentially to a particular target and does not bind in a significant amount to other components present in a test sample. Specific binding to a target under such conditions can require a binding moiety that is selected for its specificity for a particular target. A variety of assay formats can be used to select binding regions that are specifically reactive with a particular analyte. Typically, a specific or selective reaction will be at least twice the background signal or noise and more typically more than 10 times the background signal or noise.

### Monomers

The term “monomers” as used herein refers to individual nucleic acid oligomers. Two or more distinct species of nucleic acid monomers can be utilized in the methods described herein. In the methods described herein, the monomers can be, for example, RNA, DNA or RNA-DNA hybrid monomers. Each monomer species typically comprises at least one region that is complementary to a portion of another monomer species. However, the monomers are designed such that they are kinetically trapped and the system is preferably unable to equilibrate in the absence of a molecule (e.g., an analyte) that can disrupt the secondary structure of one of the monomers. Thus, the monomers are preferably unable to activate a probe in the absence of the target analyte. Hybridization of a monomer binding region of an analyte to a first monomer, or to an intervening monomer that in turn reacts with a first monomer, initiates a reaction of kinetic escapes by the monomer species resulting in activation of the probe (strand displacement).

Typically, each monomer comprises at least one region that is complementary to at least one portion of another nucleic acid for the detection schemes described herein. The makeup of the monomers for some embodiments is described in more detail below. In some embodiments, a monomer is able to interact with another molecule (e.g., an analyte or another monomer) such that an additional binding region contained in the monomer is exposed (e.g., a region that can bind to a second monomer or to at least a portion of a toe-hold region of the probe). In some embodiments the monomer can comprise a sticky end. For example, the analyte binding region of a monomer can comprise a sticky end. Other embodiments of the monomer comprise a recognition molecule that binds or interacts with an analyte. In some embodiments a monomer can comprise an aptamer that recognizes a specific molecule, and the aptamer can comprise the analyte binding region. Interaction of the analyte with an analyte binding region or to the recognition molecule of the monomer can initiate a detection process that leads to the displacement of the strands of the probe. An “activated monomer” can refer to a monomer that is bound to a target analyte or to another monomer. In some embodiments, the monomer can be linked to a recognition molecule.

“Metastable monomers” refer to monomers that, in the absence of an analyte, are kinetically disfavored from associating with other monomers comprising complementary regions.

In some embodiments, one or more monomer species are employed that have a hairpin structure. The term “hairpin” and refers to a structured formed by intramolecular base pairing in a single-stranded polynucleotide ending in an unpaired loop. A “hairpin loop” refers to a single stranded region that loops back on itself and is closed by a single base pair. In some embodiments, the monomer species employed have a RNA hairpin structure.

The term “sticky end” refers to a nucleic acid sequence that is available to hybridize with a complementary nucleic acid sequence. A “sticky end” is located at an end of a double-stranded nucleic acid. The secondary structure of the “sticky end” is preferably such that the sticky end is available to hybridize with a complementary nucleic acid under the appropriate reaction conditions without undergoing a conformational change. In some embodiments the sticky end is preferably a single stranded nucleic acid. In some embodiments, a probe can comprise a sticky end. In some embodiments, a “sticky end” can be a toe-hold region of a probe. In other embodiments, a hairpin monomer can comprise a sticky end.

### Moieties

In some embodiments, the probes can contain moieties. As used herein, the term “moiety” refers to one or more molecules that can be attached to a probe (e.g., attached to a nucleotide) and can typically be detected. Examples of moieties include, but are not limited to, “labels,” and “signal altering moieties.” In certain embodiments, a label can be a moiety that produces a signal or that interacts with another moiety to produce a signal. In certain embodiments, a label can interact with another moiety to modify a signal of the other moiety. In certain embodiments, a label can bind to another moiety or complex that produces a signal or that interacts with another moiety to produce a signal. In certain embodiments, the label emits a detectable signal when the probe is bound to a complementary target nucleic acid sequence. In certain embodiments, the label emits a detectable signal upon strand displacement of the duplex region of the probe.

In some embodiments, the probes contain one or more pairs of moieties in the duplex region of the probe. A “pair of moieties” or an “interactive label pair” refers to a pair of labels wherein at least one label exhibits a measurable characteristic upon activation of the probe (e.g., by binding of the probe to an analyte). For example, the duplex region can contain a first moiety attached to a first nucleotide and a second moiety attached to a second nucleotide that hybridizes to the first nucleotide. In the absence of a target, the probe exists predominantly in a closed conformation, with the probe forming a duplex region, and thus bringing one or more pairs of moieties in closer proximity for effective interaction, including, but not limited to, interaction between a molecular energy transfer pair or enzyme-inhibitor pair. In some embodiments, the duplex region can contain multiple pairs of moieties.

In general, upon binding to a target analyte, the interactions between the probe and the target analyte shift the equilibrium predominantly towards to an open conformation. In this open conformation, the two strands forming the duplex region are displaced from each other, thus generating a change in detectable signal from a moiety pair that can be used to detect or quantitate the target analyte. It will be understood that the labels can consist of multiple signal altering moieties if so desired.

A variety of signal altering moieties are suitable for use in the probe. For example, signal altering moieties can include a wide range of energy donor and acceptor molecules to construct resonance energy transfer probes. Energy transfer can occur, for example, through fluorescence resonance energy transfer, bioluminescence energy transfer, or direct energy transfer. Fluorescence resonance energy transfer occurs when part of the energy of an excited donor is transferred to an acceptor fluorophore which re-emits light at another wavelength or, alternatively, to a quencher group that typically emits the energy as heat. There is a great deal of practical guidance available in the literature for selecting appropriate donor-acceptor pairs for particular probes, as exemplified by the following references: Pesce et al., Eds., Fluorescence Spectroscopy (Marcel Dekker, New York, 1971); White et al., Fluorescence Analysis: A Practical Approach (Marcel Dekker, New York, 1970); and the like. The literature also includes references providing exhaustive lists of fluorescent and chromogenic molecules and their relevant optical properties, for choosing reporter-quencher pairs (see, for example, Berlman, Handbook of Fluorescence Spectra of Aromatic Molecules, 2nd Edition (Academic Press, New York, 1971); Griffiths, Colour and Constitution of Organic Molecules (Academic Press, New York, 1976); Bishop, Ed., Indicators (Pergamon Press, Oxford, 1972); Haugland, Handbook of Fluorescent Probes and Research Chemicals (Molecular Probes, Eugene, 1992) Pringsheim, Fluorescence and Phosphorescence (Interscience Publishers, New York, 1949); and the like. Further, there is extensive guidance in the literature for derivatizing acceptor and quencher molecules for covalent attachment via readily available reactive groups that can be added to a molecule. Many donor and acceptor molecules, in addition to synthesis techniques, are also readily available from many synthesis companies, such as Biosearch Technologies.

In certain embodiments, the first signal altering moiety is a fluorophore and the second signal altering moiety is a fluorescence quencher. In the absence of a target analyte, the probe is predominately in a closed conformation. Thus, the two signal altering moieties are close enough in space for effective molecular energy transfer and the fluorescent signal of the fluorophore is substantially suppressed by the fluorescence quencher. In the presence of a target analyte, the interactions between the target analyte and the probe change the conformation of the probe into an open state. Thus, the two signal altering moieties are far apart from each in space and the fluorescent signal of the fluorophore is restored for detection.

In alternative embodiments, the first signal altering moiety and the second signal altering moieties are both fluorophores that emit a certain wavelength when in close proximity and a different wavelength when further apart.

Suitable fluorophores include, but are not limited to, Alexa Fluor dyes (Invitrogen), coumarin, fluorescein (e.g., 5-carboxyfluorescein (5-FAM), 6-carboxyfluorescein (6-FAM), 2′,4′,1,4,-tetrachlorofluorescein (TET), 2′,4′,5′,7′,1,4-hexachlorofluorescein (HEX), and 2′,7′-dimethoxy-4′,5′-dichloro-6-carboxyfluorescein (JOE)), Lucifer yellow, rhodamine (e.g., tetramethyl-6-carboxyrhodamine (TAMRA), and tetrapropano-6-carboxyrhodamine (ROX)), 4,4-difluoro-5,7-dimethyl-4-bora-3a,4a-diaza-s-indacene (BODIPY), DABSYL, DABCYL, cyanine (e.g., Cy3, Cy5, and Cy7), eosine, Texas red, ROX, quantum dots, anthraquinone, nitrothiazole, and nitroimidazole compounds, Quasar and Cal-fluor dyes, and dansyl derivatives. Combination fluorophores such as fluorescein-rhodamine dimmers are also suitable (Lee et al. (1997) Nucleic Acids Res. 25:2816). Exemplary fluorophores of interest are further described in WO 01/42505 and WO 01/86001. Fluorophores can be chosen to absorb and emit in the visible spectrum or outside the visible spectrum, such as in the ultraviolet or infrared ranges.

A fluorescence quencher is a moiety that, when placed very close to an excited fluorophore, causes there to be little or no fluorescence. Suitable quenchers described in the art include, but are not limited to, BLACK HOLE QUENCHERS™ (Biosearch Technologies), Iowa Black RQ, Iowa Black FQ, rhodamine, tetramethyl rhodamine, pyrene butyrate, eosine nitrotyrosine, ethidium, fluorescein, Malachite green, Texas Red, and DABCYL and variants thereof, such as DABSYL, DABMI and Methyl Red. Fluorophores can also be used as quenchers, because they tend to quench fluorescence when touching certain other fluorophores. Suitable quenchers can be, for example, either chromophores such as DABCYL or malachite green, or fluorophores that do not fluoresce in the detection range when the detection oligonucleotide segment is in the open conformation. Gold nanoparticles, for example, are also suitable as fluorescent quenchers.

Alternatively, labels may provide antigenic determinants, radioactive isotopes, non-radioactive isotopes, nucleic acids available for hybridization, altered fluorescence-polarization or altered light-scattering. Still other labels include those that are chromogenic, chemiluminescent or electrochemically detectable. In the absence of a target analyte, these labels are preferably unavailable for detection or can be detected at a different level than after the moiety pair is disrupted.

Methods available to label a probe will be readily apparent to those skilled in the art. In some embodiments, a nucleic acid base can be labeled with a moiety. For example, a first nucleic acid base can be labeled with a first moiety and a second nucleic acid base can be labeled with a second moiety. The first and second moieties can comprise a moiety pair (e.g., a fluorophore and a quencher). In some embodiments, the first and second nucleic acid bases are hybridized to each other, for example, in the duplex region of the probe. In some embodiments, the first and second nucleic acid bases containing the moiety pair are adjacent to each other. In various embodiments, a moiety can comprise multiple molecules. For example, a moiety can comprise multiple quenchers.

In some embodiments, the duplex region of the probe comprises one moiety pair (e.g., a fluorophore and a quencher). In some embodiments, the moiety pair is located at the end of the duplex region that is at the opposite end of the toe-hold region (see, for example, FIG. 1). In some embodiments, the duplex region of the probe comprises multiple moiety pairs. For example, in some embodiments, each base pair of the duplex region can include a moiety pair.

Detection of signals and changes in signals can be carried out by any method known in the art. For example, detection of fluorescence can be carried out by any method known in the art, including, but not limited to, fluorescence microscopy, single- or multiple-photon microscopy, time-resolved fluorescence microscopy, fluorescence endoscopy, and fluorimetry.

### Methods for Detecting Analytes

Some embodiments disclosed herein relate to methods for detecting analytes using probes described herein.

In some embodiments, a probe is utilized as illustrated in FIG. 1. The small letters represent sequence segments. Letters marked with an asterisk (*) are complementary to the corresponding unmarked letter. Circles represent quenchers and stars represent fluorophores.

In the depicted embodiment, the probe comprises two nucleic acid strands, X and Y. Strand X comprises a sequence a-b and strand Y comprises a sequence a*, where region a* of strand Y is complementary to region a of strand X. In the absence of the analyte, strands X and Y form a stable structure or closed conformation in which a duplex region is formed by the hybridization region a on strand X with region a* on strand Y. The duplex region contains a moiety pair, M1 and M2. In preferred embodiments, the moiety pair is located at the opposite end of the toe-hold region of the probe. In the depicted embodiment, M1 represents a fluorescent moiety and M2 represent a quencher moiety. In the absence of an analyte (e.g., an mRNA target), M1 is quenched by M2 because M1 and M2 are in close proximity when the probe is in the closed conformation. In the depicted embodiment, strand X extends beyond strand Y to form a toe-hold region b.

The mRNA target in the depicted embodiment includes regions a* and b* that are complementary to regions a and b, respectively, of strand X of the probe. In the presence of the mRNA target, region b* of the mRNA target hybridizes to the toe-hold region b of strand X of the probe. This induces a toe-hold mediated strand displacement reaction where strand Y is displaced from the probe and the regions a*-b* of the mRNA target hybridize to regions a-b of strand X. This results in a detectable change in fluorescent signal because the probe is in an open conformation where moieties M1 and M2 are spatially separated and no longer in close proximity, and the fluorescent moiety M1 is no longer quenched.

In some embodiments, a first signal can be detected when the probe is in the closed conformation and a second signal can be detected when the strands of the probe have been displaced. In some embodiments, the presence of an analyte can be detected if the second signal is different (e.g., greater than or less than) than the first signal.

In some embodiments, the probe binds the target (e.g., target mRNA) via the following sequence of events: 1) Nucleation: The exposed single-stranded toe-hold can promote the rapid nucleation with the complementary target via base-pairing or hybridization. The single-stranded toehold typically also base-pair non-specifically to non-cognate targets in a manner identical to conventional standard single-stranded probes. 2) Branch migration: Following nucleation, probes that are bound to the cognate target begin a branch migration in which base pairs within the probe duplex region are replaced one-by-one with probe/target base pairs. Typically, no free energy is gained or lost during this branch migration process provided that the target is perfectly complementary to the probe sequence. Every mismatch can produce a large (several-kT) thermodynamic barrier to further migration. The probe duplex region can thus acts as a tunable sequence filter capable of ensuring enhanced specificity compared to conventional single-stranded probes or molecular beacons. Increasing the length of the duplex region typically increases the stringency of the specificity filter. 3) Full base-pairing: The branch migration completes successfully if the probe is bound specifically to its cognate target. If the quencher and fluorophore pair are located at the end of the probe opposite the toehold, then a fluorescent signal is generated only upon displacement of the quencher strand following the stringent specificity check posed by the branch migration process.

In some embodiments, a probe is utilized as illustrated in FIG. 2. In the depicted embodiment, the probe comprises two nucleic acid strands, A and B. Strand A comprises a sequence a-x-b and strand B comprises a sequence x*, where region x* is complementary to region x. In the absence of the analyte, strands A and B form a stable structure or closed conformation in which a duplex region is formed by the hybridization region x on strand A with region x* on strand B. In the depicted embodiments, the duplex region contains multiple moiety pairs. In the depicted embodiment, strand A contains multiple fluorescent moieties (closed stars), and strand B contains multiple quencher moieties (circles). In the absence of an analyte (e.g., an mRNA target), the fluorescent moieties are quenched by the quencher moieties because the fluorescent moieties and the quencher moieties are in close proximity when the probe is in the closed conformation. In the depicted embodiment, strand A extends beyond strand B to form toe-hold regions a and b.

The mRNA target T in the depicted embodiment includes regions a*-x*-b* that are complementary to regions a-x-b, respectively, of strand A of the probe. In the presence of the mRNA target T, a strand displacement reaction occurs where strand B is displaced from the probe and the regions a*-x*-b* of the mRNA target T hybridize to regions a-x-b of strand A. The toe-hold mediated strand displacement reaction can be initiated either by region b* of the mRNA target hybridizing to toe-hold region b of strand A of the probe or by region a* of the mRNA target hybridizing to toe-hold region a of strand A of the probe. The strand displacement reaction results in a detectable change in fluorescent signal because the probe is in an open conformation where the fluorescent moieities and quencher moieities are spatially separated and no longer in close proximity, and the fluorescent moieties are no longer quenched.

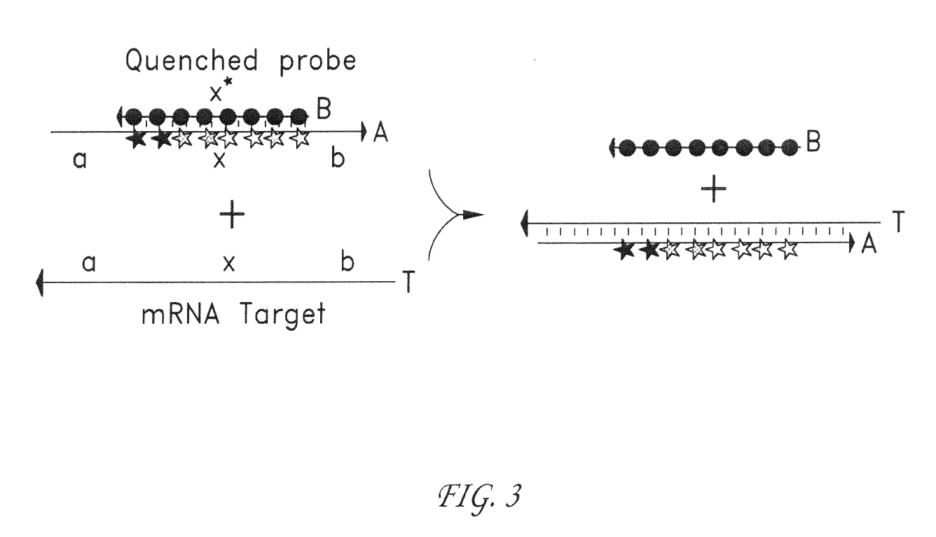

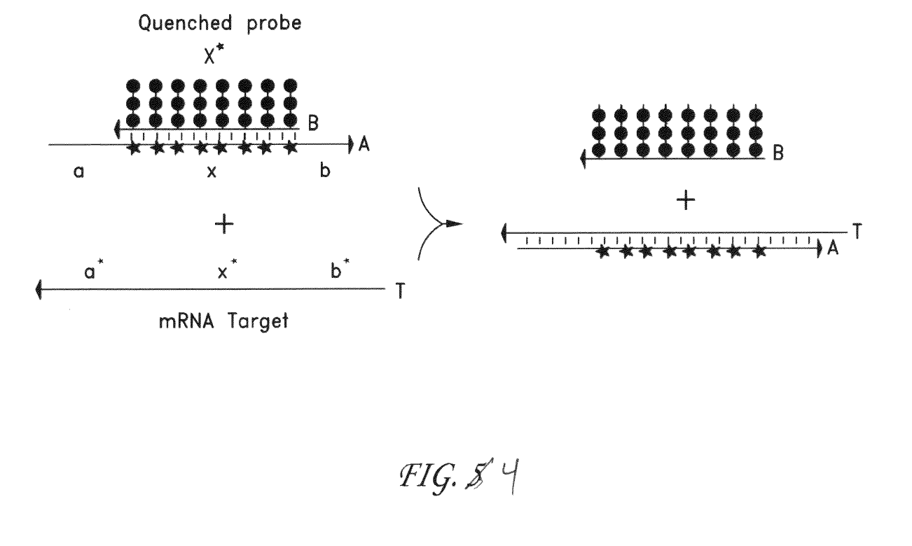

The use of multiple fluorophores, as depicted in FIG. 2, can render quantitative amplification of the analyte or target signal. For example, if k number of fluorophores are used, a k-fold signal amplification will result when the fluorophores are separated from the quenchers. In some embodiments, multiple spectrally indistinct fluorophores can be included in the probe. In other embodiments, spectrally distinct combinations of fluorophores can be included in the probe, as illustrated in FIG. 3. Spectrally distinct fluorophores (depicted as stars with distinct shading patterns in FIG. 3) render multiplexed amplification. With ultrahigh resolution light microscopy (Rice J H (2007) Molecular Systems 3:781-793) where the spatial co-localizaiton of fluorophores can be detected, k fluorophores can generate 2^{k-1} distinct barcode-type readouts. This multiplexing can be used, for example, to identify multiple targets in a sample based upon the readout (e.g., fluorescence pattern) for each target.

In further embodiments, multiple repeats of one or more moiety pairs (e.g., fluorophore/quencher pairs) can be included in the probe. Each repeat can be separated by one or more base pairs. In various embodiments, either or both strands of the probe can contain bulge loops. The bulge loops can facilitate the strand displacement reaction, for example, by acting as thermal ratchets. In some embodiments, bulge loops can facilitate the strand displacement of longer hybridized sequences.

In some embodiments a probe is utilized as illustrated in FIG. 4. In the depicted embodiment, the probe comprises two nucleic acid strands, A and B. Strand A comprises a sequence a-x-b and strand B comprises a sequence x*, where region x* is complementary to region x. In the absence of the analyte, strands A and B form a stable structure or closed conformation in which a duplex region is formed by the hybridization region x on strand A with region x* on strand B. In the depicted embodiments, the duplex region contains multiple moiety pairs. In the depicted embodiment, strand A contains multiple fluorescent moieties (closed stars), and strand B contains multiple quencher moieties (closed circles). Furthermore, in the depicted embodiment, each base in strand A contains multiple quenchers attached to it, which can increase the quenching efficiency of the fluorophores (see, e.g., C. J. Yang et al. 2005 J. Am. Chem. Soc. 127:12772-12773, which is herein incorporated by reference in its entirety). For example, the branching base can branch an abasic backbone of the nucleic acid chain, with multiple quenchers attached to it. In the absence of an analyte (e.g., an mRNA target), the fluorescent moieties are quenched by the quencher moieties because the fluorescent moieties and the quencher moieties are in close proximity when the probe is in the closed conformation. In the depicted embodiment, strand A extends beyond strand B to form toe-hold regions a and b.

The mRNA target T in the depicted embodiment includes regions a*-x*-b* that are complementary to regions a-x-b, respectively, of strand A of the probe. In the presence of the mRNA target T, a strand displacement reaction occurs where strand B is displaced from the probe and the regions a*-x*-b* of the mRNA target T hybridize to regions a-x-b of strand A. The toe-hold mediated strand displacement reaction can be initiated either by region b* of the mRNA target hybridizing to toe-hold region b of strand A of the probe or by region a* of the mRNA target hybridizing to toe-hold region a of strand A of the probe. The strand displacement reaction results in a detectable change in fluorescent signal because the probe is in an open conformation where the fluorescent moieities and quencher moieities are spatially separated and no longer in close proximity, and the fluorescent moieties are no longer quenched.

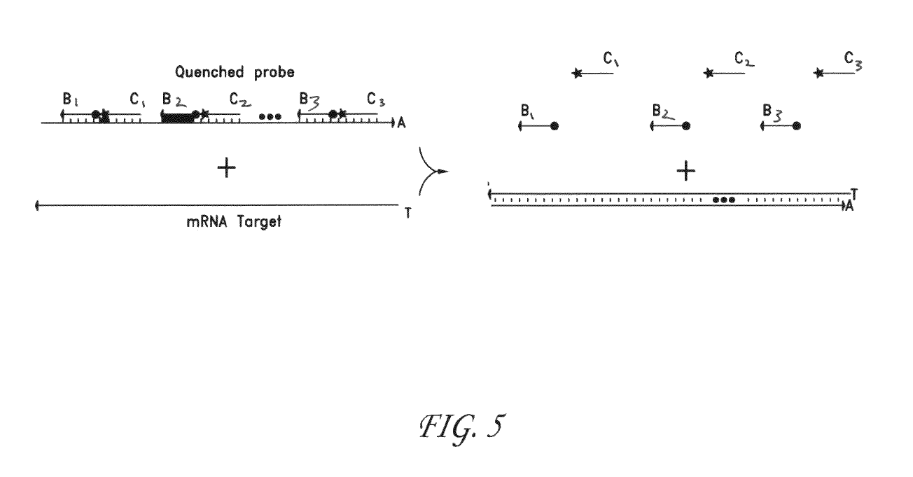

In some embodiments, a probe is utilized as illustrated in FIG. 5. In some embodiments, the probe comprises at least three nucleic acid strands, A, B1, and C1. In the depicted embodiment, the probe comprises nucleic acid strands A, B1, B2, B3, C1, C2, and C3. Strand A does not contain a signal moiety. Strands B1, B2, and B3 each include one or more moieties (e.g., quenchers in the depicted embodiment) at one end of each strand. Strands C1, C2, and C3 each include one or more moieties (e.g., fluorophores in the depicted embodiment) at one end of each strand, such that the moieties on B1 and C1 form a moiety pair, the moieties on B2 and C2 form a moiety pair, and the moieties on B3 and C3 form a moiety pair. Strands B1, B2, B3, C1, C2, and C3 are each complementary to a different region of strand A. In the absence of the analyte, strands A, B1, B2, B3, C1, C2, and C3 form a stable structure or closed conformation in which B1, B2, B3, C1, C2, and C3 are hybridized to strand A. In the absence of an analyte (e.g., an mRNA target), the fluorescent moiety on C1 is quenched by the quencher moiety on B1, the fluorescent moiety on C2 is quenched by the quencher moiety on B2, and the fluorescent moiety on C3 is quenched by the quencher moiety on B3, because the fluorescent moieties and the quencher moieties are in close proximity when the probe is in the closed conformation.

In the presence of the mRNA target T, a strand displacement reaction occurs where strands B1, B2, B3, C1, C2, and C3 are displaced from strand A and the mRNA target T hybridizes to strand A. In some embodiments, the strand displacement reaction is toe-hold mediated. In some embodiments, the strand displacement reaction is initiated by the binding of one or more regions of the mRNA target T to one or more single-stranded regions of strand A of the probe when the probe is in the closed conformation. The strand displacement reaction results in a detectable change in fluorescent signal because the probe is in an open conformation where the fluorescent moieities and quencher moieities are spatially separated and no longer in close proximity, and the fluorescent moieties are no longer quenched. An advantage of the embodiment depicted in FIG. 4 and its variations is that end modifications are used for attaching the moiety (e.g., fluorophore or quencher) to the nucleic acid strands. In addition, the end-to-end placement of a moiety pair (e.g., fluorophore/quencher) provides high quenching efficiency when the probe is in the closed state.

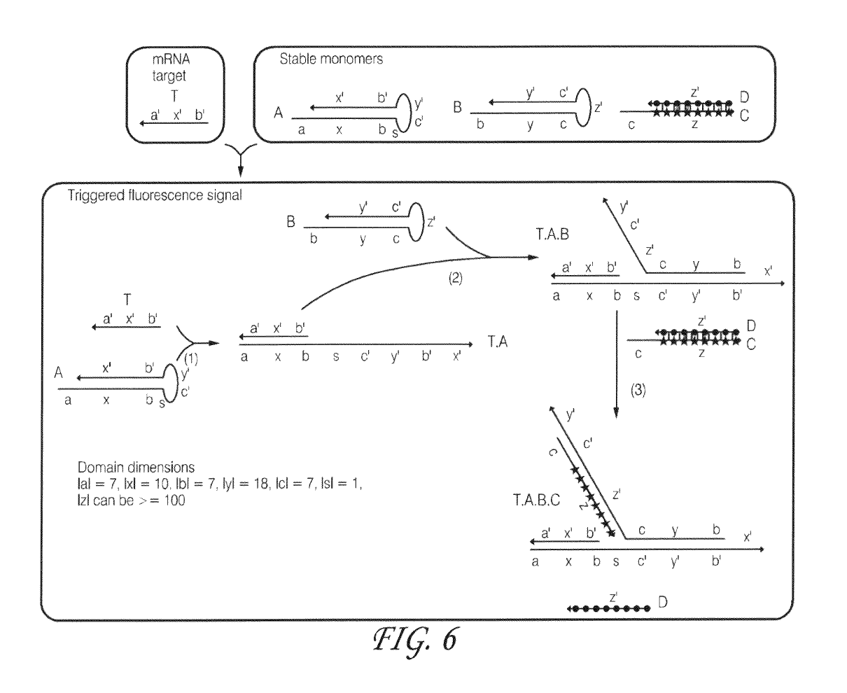

In some embodiments, a probe and monomers are utilized as illustrated in FIG. 6 to generate a detectable signal or a detectable change in signal in the presence of an analyte. In the depicted embodiment, an analyte T interacts with and opens a first monomer A, that in turn reacts with a second monomer B, that in turn reacts with the probe to displace the strands of the probe, thereby generating a detectable signal or a detectable change in signal. In some embodiments, a probe can be used in combination with a single monomer to detect a target analyte. For example, the analyte can bind to the monomer. Upon the analyte binding to the monomer, a region of the monomer can become available to bind to and activate the probe, leading to a change in signal detection.

In the depicted embodiment, the probe comprises two nucleic acid strands, C and D. Strand C comprises a sequence z-c and strand B comprises a sequence z*, where region z* is complementary to region z. In the absence of the analyte, strands C and D form a stable structure or closed conformation in which a duplex region is formed by the hybridization region z on strand C with region z* on strand D. In the depicted embodiments, the duplex region contains multiple moiety pairs. In the depicted embodiment, strand C contains multiple fluorescent moieties (closed stars), and strand D contains multiple quencher moieties (closed circles). In some embodiments, the duplex region contains one pair of moieties. In the absence of an analyte (e.g., an mRNA target), the fluorescent moieties are quenched by the quencher moieties because the fluorescent moieties and the quencher moieties are in close proximity when the probe is in the closed conformation. In the depicted embodiment, strand C extends beyond strand D to form toe-hold region c.

In the embodiment depicted in FIG. 6, monomers A and B are further utilized in the detection scheme. In the absence of the analyte, the probe, monomer A, and monomer B co-exist metastably. Monomers A and B preferably comprise hairpin monomers and preferably each comprises a sticky end, a hairpin loop region at the opposite end of the sticky end, and two “stem regions,” a first stem region and a second stem region, that together can form a duplex region.

In FIG. 6, monomer A comprises an analyte binding (complementarity) region comprising sequence a-x-b and a second monomer binding (complementarity) region comprising sequence c*-y*-b*.

The mRNA target T in the depicted embodiment includes regions a*-x*-b* that are complementary to regions a-x-b, respectively, of monomer A. Preferably, upon hybridization of the analyte T to the sticky end of monomer A, one arm of the hairpin structure is displaced. This opens the hairpin. In the depicted embodiment, in the presence of an analyte T, the analyte T nucleates at the sticky end a of monomer A by pairing segment a* with a. This induces a strand displacement interaction resulting in the hybridization of the regions a*-x*-b* of analyte T with regions a-x-b, respectively, of monomer A resulting in the formation of complex T.A (step (I) in FIG. 6). In the depicted embodiment, T.A has a newly exposed single-stranded tail that contains the sequence s-c*-y*-b*-x*.

In the depicted embodiment, the single-stranded tail of the T.A complex nucleates at the sticky end b of monomer B by pairing segment b* with b. This induces a strand displacement interaction resulting in the hybridization of the regions b*-y*-c* of T.A with regions b-y-c, respectively, of monomer B, opening up monomer B and resulting in the formation of complex T.A.B (step (2) in FIG. 6).

In the depicted embodiment, a single-stranded region of the T.A.B complex nucleates at the toe-hold c of strand C of the probe by pairing segment c* with c. This induces a strand displacement interaction resulting in the hybridization of regions c*-z* of T.A.B with regions c-z, respectively, of strand C, displacing strand D from the probe and resulting in the formation of complex T.A.B.C (step (3) in FIG. 6). The strand displacement reaction results in a detectable change in fluorescent signal because the probe is in an open conformation where the fluorescent moieities and quencher moieities are spatially separated and no longer in close proximity, and the fluorescent moieties are no longer quenched.

Through the transduction by hairpin monomer A and hairpin monomer B as depicted in FIG. 6, the analyte T and the fluorophore strand C share no common sequence. Schemes such as those depicted in FIG. 6, thus permit the same quenched probe nucleic acid strand (strands C and D) to be reused for different targets, while utilizing additional monomer pairs designed to detect the additional targets.

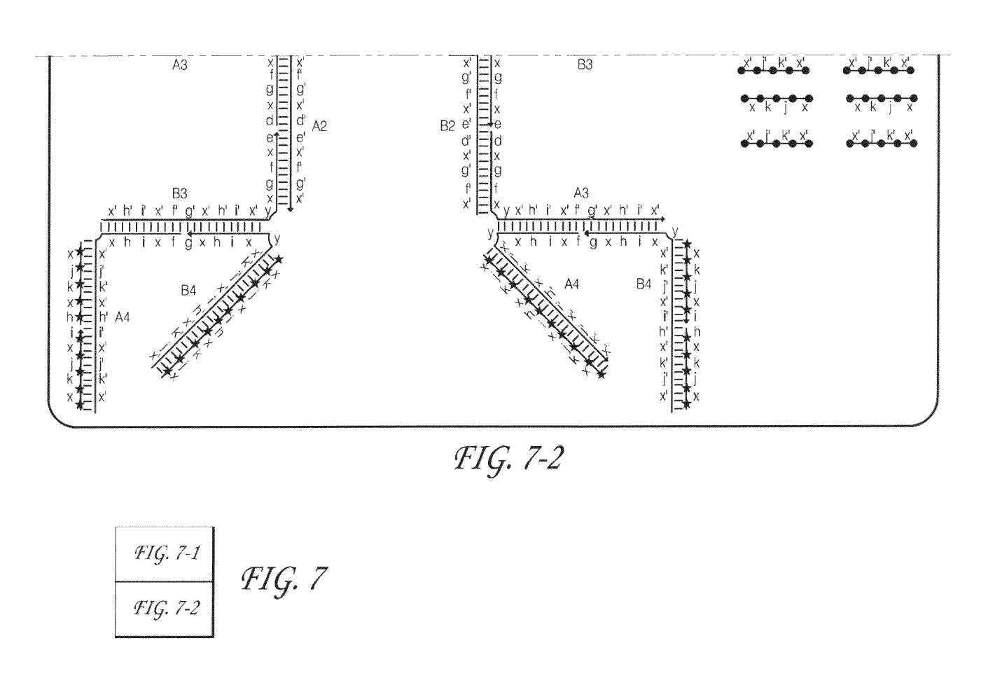

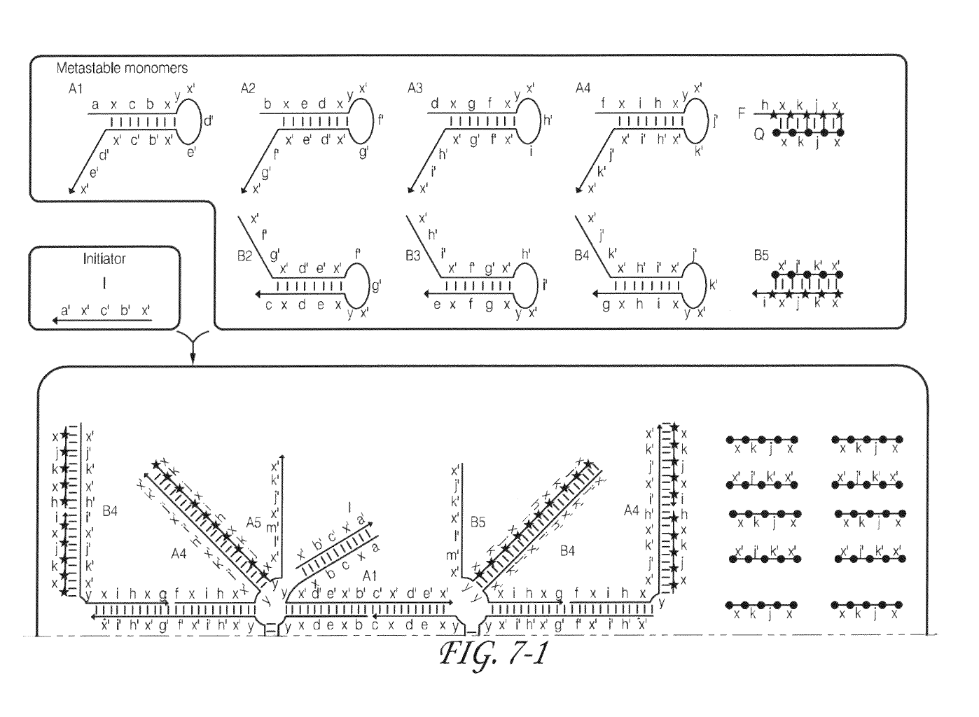

In some embodiments, one or more probes and multiple monomers are utilized as to generate an amplified detectable signal or an amplified detectable change in signal in the presence of an analyte. For example, FIG. 7 illustrates an embodiment of a nucleated dendritic amplification process that occurs in the presence of a target.

### Delivery of Probes, Monomers, and Accessory Molecules to Target Cells

Probes, monomers, and any accessory molecules, such as, for example, helper molecules, can be formulated with any of a variety of carriers well known in the art to facilitate introduction into a sample (e.g., cells). Suitable carriers for delivery of nucleic acids to cells are well known in the art and include, for example, polymers, proteins, carbohydrates and lipids. For example, a cyclodextrin-containing polymer can be used for the delivery of the probes, monomers, and/or any accessory molecules. Commercial transfection reagents known in the art, such as, for example, LNCaP (Altogen Biosystems) or lipofectamine (Invitrogen), can be used.

Delivery of nucleic acids can be accomplished, for example, as described by Heidel (Heidel, J. D. 2005. Targeted, systematic non-viral delivery of small interfering RNA in vivo. Doctoral thesis, California Institute of Technology. 128p., herein incorporated by reference in its entirety). Also contemplated within the scope of the subject matter are gene delivery systems as described by Felgner et al. (Feigner et al. 1997. Hum Gene Ther 8:511-512, herein incorporated by reference in its entirety), including cationic lipid-based delivery systems (lipoplex), polycation-based delivery systems (polyplex) and a combination thereof (lipopolyplex). Cationic lipids are described, for example, in U.S. Pat. Nos. 4,897,355 and 5,459,127, each of the foregoing which is herein incorporated by reference in its entirety. Proteins can also be used for nucleic acid delivery, such as synthetic neoglycoproteins (Ferkol et al. 1993. FASEB J 7:1081-1091; Perales et al. 1994. Proc Nat Acad Sci 91:4086-4090; each of which is incorporated herein by reference in its entirety), epidermal growth factor (EGF) (Myers, EPO 0273085, incorporated herein by reference in its entirety), and other ligands for receptor-mediated gene transfer (Wu and Wu. 1987. J Biol Chem 262(10):4429-4432; Wagner et al. 1990. Proc Natl Acad Sci USA 87(9):3410-3414; Ferkol et al. 1993. J Clin Invest 92(5):2394-2300; Perales et al. 1994. Proc Natl Acad Sci USA 91(9):4086-4090; Myers, EPO 0273085; each of which is incorporated herein by reference in its entirety).

Viral and viral vector-like delivery systems generally known in the art, such as those described, for example, in U.S. Pat. No. 7,0333,834; U.S. Pat. No. 6,899,871; U.S. Pat. No. 6,555,367; U.S. Pat. No. 6,485,965; U.S. Pat. No. 5,928,913; U.S. patent application Ser. No. 10/801,648; U.S. patent application Ser. No. 10/319,074, and U.S. patent application Ser. No. 09/839,698, each of which is herein incorporated by reference, are also contemplated for use in the present subject matter. In addition, standard electroporation techniques can be readily adopted to deliver probes, monomers, and/or any accessory molecules.

Delivery of probes, monomers, and/or any accessory molecules can occur in vivo or ex vivo. In some embodiments, cells can be removed from a patient and transfected with the probes, monomers, and/or any accessory molecules. In other embodiments, probes, monomers, and/or any accessory molecules can be delivered to cells in vivo such as by, for example, injection of the probes, monomers, and/or any accessory molecules within a delivery vehicle into the bloodstream or by intramuscular, subcutaneous, or intraperitoneal means. An appropriate means of delivering probes, monomers, and/or any accessory molecules to a desired population of cells can be identified by the skilled practitioner based on the particular circumstances without undue experimentation.

### Diagnostic Applications

Embodiments disclosed herein relate to diagnostic and prognostic methods for the detection of a disease or disorder and/or monitoring the progression of a disease or disorder. As used herein, the phrase “diagnostic” refers identifying the presence of or nature of a disease or disorder. The detection of an analyte (e.g., an mRNA) associated with a disease or disorder) provides a means of diagnosing the disease or disorder. Such detection methods may be used, for example, for early diagnosis of the condition, to determine whether a subject is predisposed to a disease or disorder, to monitor the progress of the disease or disorder or the progress of treatment protocols, to assess the severity of the disease or disorder, to forecast the an outcome of a disease or disorder and/or prospects of recovery, or to aid in the determination of a suitable treatment for a subject. The detection can occur in vitro or in vivo.

Diseases contemplated for diagnosis in embodiments described herein include any disease in which an analyte, such as an analyte associated with the disease, is present in a cell and can initiate strand displacement of the probe. Preferred embodiments include, but are not limited to, diseases in which the analyte is a nucleic acid molecule. In some embodiments, the nucleic acid molecule is an mRNA molecule associated with a disease or disorder, such as a mutant mRNA molecule. However, disease-associated analytes can be, for example and without limitation, nucleic acid sequences, proteins, peptides, lipids, carbohydrates and small molecules.

In some embodiments, the disease to be diagnosed is a type of cancer, such as, for example, leukemia, carcinoma, lymphoma, astrocytoma, sarcoma and particularly Ewing's sarcoma, glioma, retinoblastoma, melanoma, Wilm's tumor, bladder cancer, breast cancer, colon cancer, hepatocellular cancer, pancreatic cancer, prostate cancer, lung cancer, liver cancer, stomach cancer, cervical cancer, testicular cancer, renal cell cancer, and brain cancer.

In other embodiments, the disease to be diagnosed is associated with infection by an intracellular parasite. For example, the intracellular parasite may be a virus such as, for example, an adenovirus, cytomegalovirus, Epstein-Barr virus, herpes simplex virus, human herpesvirus 6, varicella-zoster virus, hepatitis viruses, papilloma virus, parvovirus, polyomavirus, measles virus, rubella virus, human immunodeficiency virus (HIV), or human T cell leukemia virus. In other embodiments, the intracellular parasite may be a bacterium, protozoan, fungus, or a prion. More particularly, the intracellular parasite can be, for example, Chlamydia, Listeria, Salmonella, Legionella, Brucella, Coxiella, Rickettsia, Mycobacterium, Leishmania, Trypanasoma, Toxoplasma, and Plasmodium.

In some embodiments, a probe may be activated, either in vitro or in vivo, in response to the presence of an analyte. Thus, methods disclosed herein can be used for detecting the presence of an analyte in a sample or cells. In some embodiments, the methods can be used in forensics, environmental, and commercial applications.

### Compositions and Kits for Analyte Detection and Diagnosis

Compositions and kits for detecting the presence of an analyte are contemplated for use within the scope of the subject matter. In some embodiments, the compositions and/or kits comprise a probe (e.g., a quenched probe) and instructions for use. In some embodiments, the compositions and/or kits comprise a probe, one or more nucleic acid monomers (e.g., hairpin monomers), and instructions for use. Upon delivery to a target cell or sample and recognition of the analyte by the probe or monomer, strand displacement of the probe is initiated causing a change in signal detection that can readily be measured. In some embodiments, the kits can comprise an aptamer that exposes a nucleic acid that binds to the probe only in the presence of the target. In some embodiments, the probe can be configured for a particular analyte. In other embodiments, a generic probe can be used in combination with one or more recognition molecules that can be configured for one or more analytes.

The compositions and/or kits can also contain other components, such as, for example, accessory molecules that facilitate analyte recognition and aid the triggering strand displacement of the probe. Accessory molecules typically comprise nucleic acid molecules. The compositions and/or kits may further comprise a reaction container, various buffers, and one or more reference samples (e.g., a negative and/or positive control).

Furthermore, the composition and/or kit can comprise a carrier that facilitates the introduction of nucleic acids, such as, for example, probes, monomers and/or accessory nucleic acid molecules, into a cell, such as a cell containing an analyte associated with a disease or disorder. Carriers for delivery of nucleic acids into cells are well known in the art and examples are described above. In some embodiments, the kit is used to deliver probes, monomers and/or accessory nucleic acid molecules to the tissues of a patient, wherein the tissues comprise cells comprising an analyte associated with a disease or disorder. In other embodiments, the kit is used to select for cells containing an analyte in vitro.

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1

This example demonstrates the generation of a self-quenched probe and gel electrophoresis of the quenched probe system using in situ hybridization conditions.

The probe was labeled with the fluorescent moiety Alexa647 on the fluorescent strand and the quencher moiety Iowa Black RQ on the quencher strand. The fluorescent strand was 50 nucleotides long (SEQ ID NO: 1) and the quencher strand was 25 nucleotides long (SEQ ID NO: 2). The fluorescent strand was 5′-labeled with Alexa647 using a six-carbon spacer and the quencher strand was 3′-labeled with Iowa Black RQ. The fluorescent strand and quencher strand were annealed (heated to 70° C. for 5 minutes, cool to RT slowly over 1.5 hour period) before they were mixed with the EGFP mRNA. The toe-hold region was 25 nucleotides in length and the duplex region was 25 bas pairs in length. All samples were incubated for 1 hour at 45° C. in hybridization solution (50% formamide, 0.1% Tween20, 9 mM citric acid (pH=6.0), and 2×SSC (300 mM NaCl, 30 mM Na₃C₆H₅O₇, pH=7.0). The samples were loaded with 10% glycerol buffer into a 1.5% native agarose gel, prepared with 1×LB buffer. The gel was run at 150V for 45 minutes and imaged using a Fuji FLA-5100 fluorescent scanner. The excitation laser sources and emission filters were: 635 nm laser with a 665 nm long pass filter (Alexa647) and a 473 nm laser with a 575 nm long pass filter (Sybr Gold).

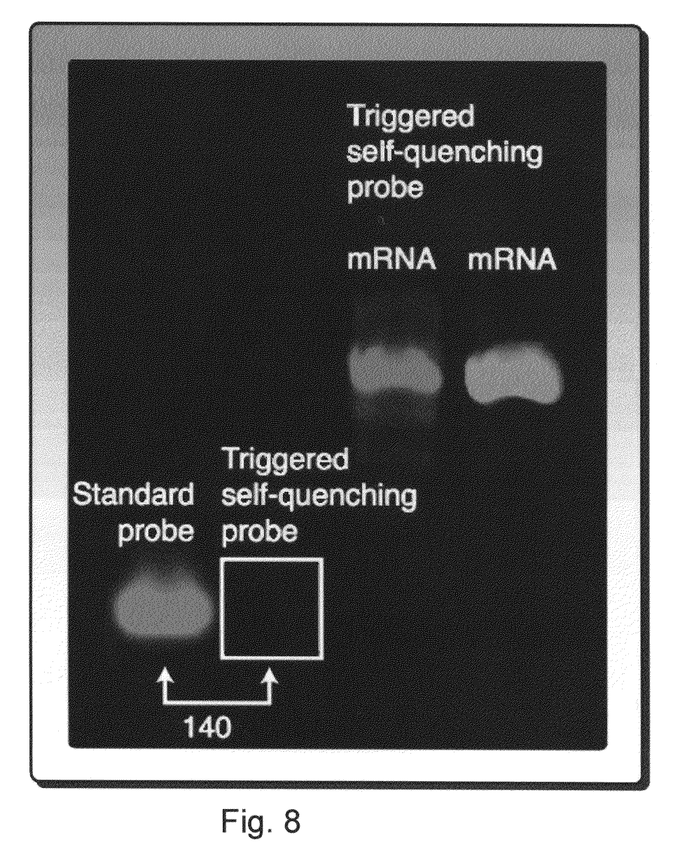

The results demonstrate active background suppression in the absence of target (FIG. 8, lane 2) and sensitive detection when mixed with full-length EGFP mRNA (FIG. 8, lane 3). A factor of 140 in reduction of the probe signal was obtained when bound to the quencher and then a 90% recovery of signal was obtained after the target mRNA was introduced. Lane 4 in FIG. 8 shows a full-length EGFP mRNA (pre-stained with Sybr Gold).

### Example 2

This example demonstrates in situ verification of active background suppression with the self-quenching probe.

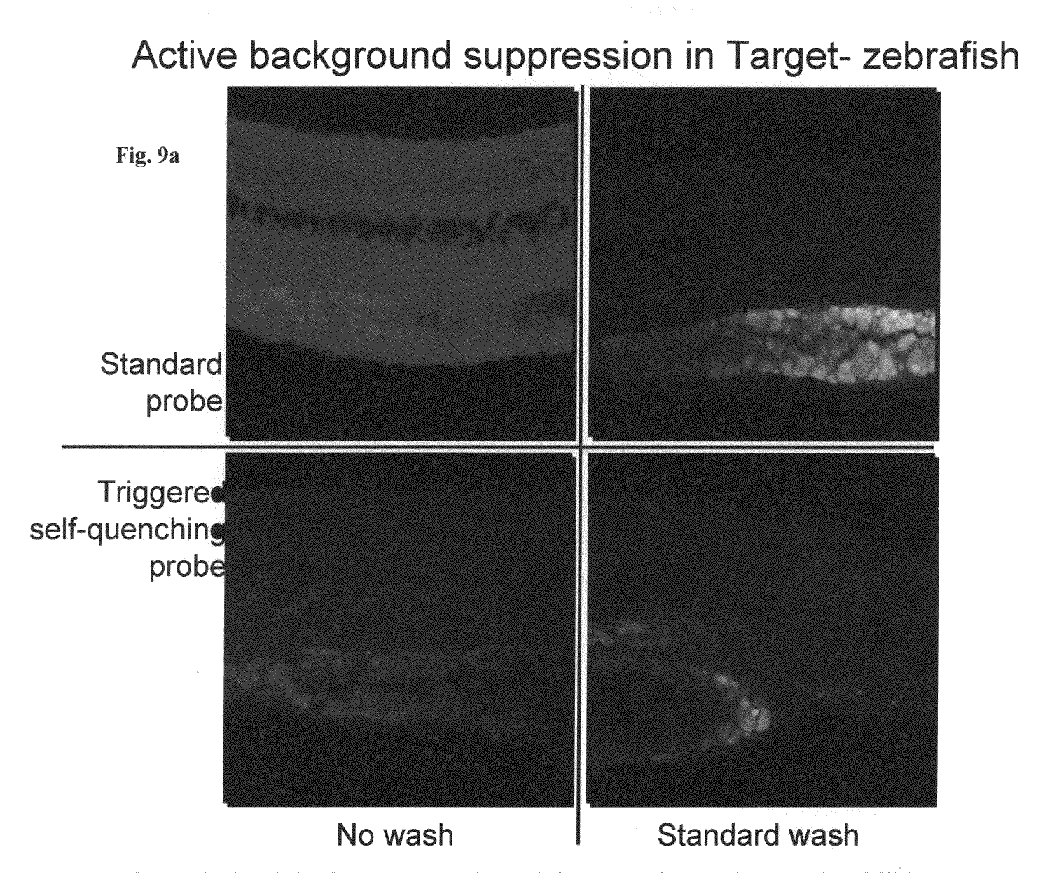

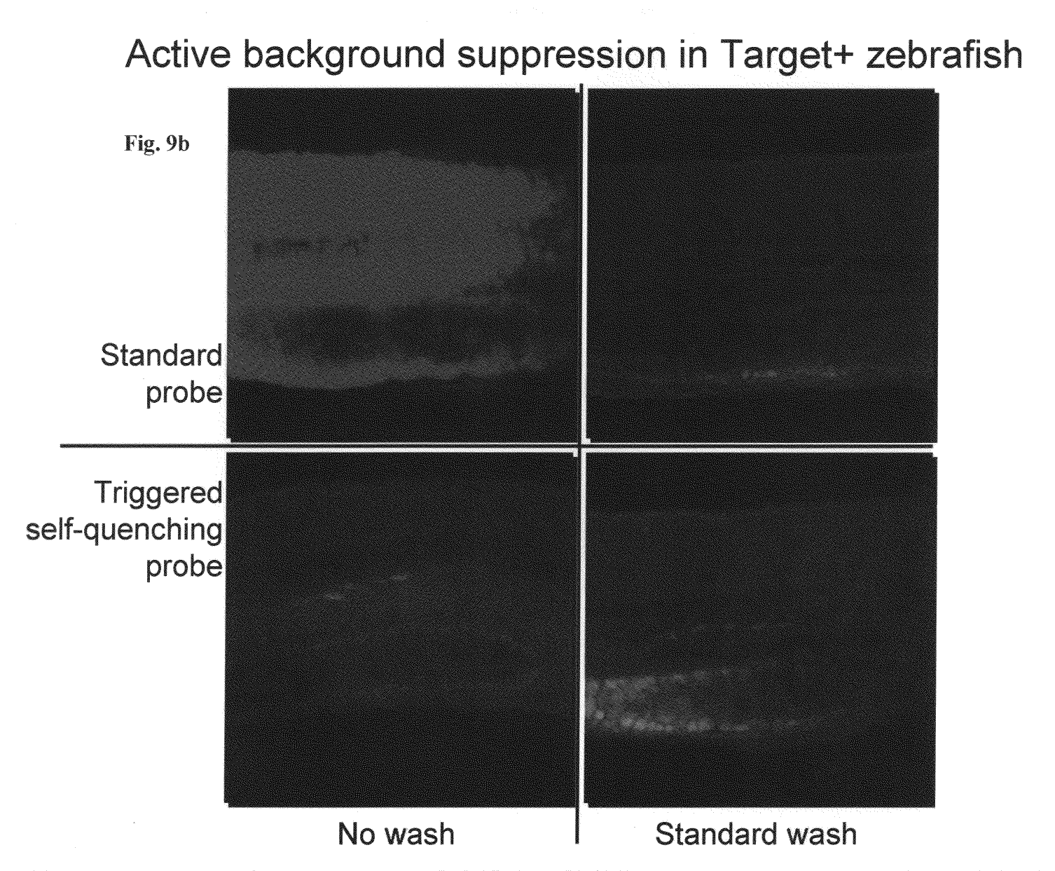

The quenched probe, as described in Example 1, was used to target an enhanced green fluorescent protein (EGFP) gene driven by an flk1 promoter in transgenic zebrafish embryos (25hpf). Without any washes, background staining (blue) of the quenched probe was significantly lower than that of a regular probe in a wildtype embryo (Target−) in which the EGFP gene is absent (FIG. 9A). With complete washes, the difference between the samples became negligible (FIG. 9A).

In the transgenic embryo (Target+), the EGFP mRNA was detected with the quenched probe without any washes but not with the regular probe due to the high background fluorescence (FIG. 9B). With complete washes, staining can was observed with both methods (FIG. 9B).

Hybridization was performed overnight (16 hours or more) at 45° C. in hybridization solution described in Example 1. In the standard wash, embryos were washed with a graded series of hybridization solution and 2×SSC at 45° C. Embryos were further washed with 1×15 minutes and 1×30 minutes 2×SSC at 45° C. Finally, the embryos were washed with a series of graded 2×SSC and PBST (1×PBS, 0.1% Tween20) solutions at room temperature. A Zeiss 510 upright confocal microscope with a LD LCI Plan-Apochromat 25×/0.8 Imm Corr DIC objective was used to acquire the images. The channel used to show the morphology of the embryos (with green false coloring) was obtained using a 488 nm Ar laser for excitation and a bandpass (BP 500-530) emission filter. The Alexa 647 channel (with blue false coloring) was acquired by exciting the fluorophores with a 633 nm HeNe laser and collecting fluorescence with a long pass (LP 650) filter.

### Example 3

For in vivo imaging in cells, one can use a quenched probe comprising a duplex region formed between a fluorescent strand and a quencher strand, a single-stranded toe-hold region on the fluorescent strand, a fluorophore on the fluorescent strand at the end opposite the toe-hold region, and a quencher on the quencher strand situated adjacent to the fluorophore. The mRNA target is a fluorescent protein transgene that is present in one cell line (Target +) and absent in another (Target −). Quenched probes are microinjected or transfected (e.g., using Oligofectamine) into the cells. In Target− cells, the quenched probe remains in the closed state with the fluorescent and quencher strands base-paired to each other and no fluorescent signal is generated. In Target+ cells, probe toe-hold nucleates with the mRNA target and the quencher strand is displaced via branch migration as the fluorescent strand base-pairs to the target mRNA. The separation of the fluorophore on the fluorescent strand and the quencher on the quencher strand generates a fluorescent signal.

### Example 4

A probe is used to detect the presence of a target in a test sample. The probe comprises two nucleic acid strands that are hybridized, one moiety pair (a fluorophore and a quencher), and a toe-hold region. The probe is contacted with a test sample and with a control sample (in which the target is absent). The strands of the probe separate in the presence of the target, generating a fluorescence signal in the test sample. This signal is greater than the fluorescence signal generated when the probe is contacted with a control sample in which the target is absent and when the strands of the probe do not separate.

While the present teachings have been described in terms of these exemplary embodiments, the skilled artisan will readily understand that numerous variations and modifications of these exemplary embodiments are possible without undue experimentation. All such variations and modifications are within the scope of the current teachings.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein.

Unless otherwise indicated, the singular use of various words, including the term “an” or “an” denotes both the option of a single or more than one. In addition, the use of the term “and/or” denotes various embodiments that include: both options, either option in the alternative, or the combination of either option in the alternative and both options. When describing various combinations, kits, probes, methods, etc., it will be understood that unless otherwise stated, the combinations are described as comprising, consisting of, and consisting essentially of. This does not apply to the claims or to situations in the specification where the term “consisting of” is used.

### INCORPORATION BY REFERENCE

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

### EQUIVALENTS

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A probe for detecting the presence of a target nucleic acid analyte in a cell, comprising:
a first nucleic acid strand;
a second nucleic acid strand hybridized to the first nucleic acid strand;
a duplex region formed between the first nucleic acid strand and the second nucleic acid strand, wherein the duplex region comprises one or more moiety pairs, wherein the moiety pair comprises a first moiety attached to a first nucleotide of the first nucleic strand and a second moiety attached to a second nucleotide of the second nucleic strand, wherein a first signal can be detected from one or more moieties of the moiety pair when the first nucleic acid strand is hybridized to the second nucleic acid strand, wherein the first signal is different than a second signal that can be detected from one or more moieties of the moiety pair when the first nucleic acid strand is not hybridized to the second nucleic acid strand; and
a first toe-hold region comprising a first single-stranded region of the first nucleic acid strand that extends beyond the second nucleic acid strand, wherein in the presence of the target nucleic acid analyte, the first nucleic strand and the second nucleic acid strand separate, such that the second signal can be detected.

2. The probe of claim 1, wherein a portion of the first toe-hold region is complementary to a portion of the target nucleic acid analyte.

3. The probe of claim 1, wherein the first toe-hold region is complementary to a first portion of a first monomer, wherein a second portion of said first monomer is complementary to a first portion of a second monomer, and wherein a second portion of said second monomer is complementary to a portion of the target nucleic acid analyte.

4. The probe of claim 1, wherein the first toe-hold region comprises a length of about 4 to about 50 nucleotides.

5. The probe of claim 1, wherein said duplex region comprises a length of about 8 to about 50 nucleotides.

6. The probe of claim 1, wherein said duplex region comprises one moiety pair.

7. The probe of claim 1, wherein said moiety pair is located in a portion of the probe that is at the opposite end from said toehold region.

8. The probe of claim 1, wherein the first moiety comprises a fluorophore and the second moiety comprises one or more quenchers.

9. The probe of claim 1, wherein the first moiety comprises a fluorophore and the second moiety comprises a fluorophore.

10. The probe of claim 1, further comprising a second toe-hold region comprising a second region of the first nucleic acid strand that extends beyond the second nucleic acid strand.

11. The probe of claim 1, wherein the duplex region comprises multiple moiety pairs.

12. The probe of claim 11, wherein each moiety pair comprises a fluorophore and a quencher.

13. The probe of claim 12, wherein said fluorophores are spectrally distinct.

14. The probe of claim 12, wherein said fluorophores are spectrally indistinct.

15. The probe of claim 11, wherein each base pair in the duplex region comprises a moiety pair.

16. A kit comprising the probe of claim 1 and instructions for use.

17. A method for detecting the presence of a target nucleic acid analyte in a sample, comprising:
contacting the sample with a probe, wherein the probe comprises:
a first nucleic acid strand;
a second nucleic acid strand hybridized to the first nucleic acid strand;
one or more moiety pairs, wherein the moiety pair comprises a first moiety attached to the first nucleic acid strand and a second moiety attached to the second nucleic acid strand, wherein a first signal can be detected from one or more moieties of the moiety pair when the first nucleic acid strand is hybridized to the second nucleic acid strand; and
a first toe-hold region comprising a first single-stranded region of the first nucleic acid strand that extends beyond the second nucleic acid strand, wherein a portion of the first toe-hold region is substantially complementary to a portion of the target nucleic acid;
wherein in the presence of said target nucleic acid analyte in the sample, the first nucleic acid strand is displaced from said second nucleic acid strand and a second signal is generated that can be detected from one or more moieties of the moiety pair; and
measuring the first signal and the second signal, detecting the presence of the target nucleic acid analyte when the second signal is different than the first signal.

18. The method of claim 17, wherein the first toe-hold region comprises a length of about 4 to about 50 nucleotides.

19. The method of claim 17, wherein said moiety pair is located in a portion of the probe that is at the opposite end from said toehold region.

20. The method of claim 17, wherein the first moiety comprises a fluorophore and the second moiety comprises one or more quenchers.

21. The method of claim 17, wherein the first moiety comprises a fluorophore and the second moiety comprises a fluorophore.

22. The method of claim 17, further comprising a second toe-hold region comprising a second single-stranded region of the first nucleic acid strand that extends beyond the second nucleic acid strand.

23. The method of claim 17, wherein the probe comprises multiple moiety pairs.

24. The method of claim 23, wherein each moiety pair comprises a fluorophore and a quencher.

25. The method of claim 24, wherein said fluorophores are spectrally distinct.

26. The method of claim 24, wherein said fluorophores are spectrally indistinct.

27. The method of claim 17, wherein the target analyte is associated with a disease or disorder.

28. The method of claim 17, wherein the target analyte is an mRNA molecule.


SEQUENCE LISTING (2 sequences)

SEQ ID NO: 1 - DNA, 50 bp, Artificial Sequence
  Strand of probe
gttcttctgcttgtcggccatgatatagacgttgtggctgttgtagttgt

SEQ ID NO: 2 - DNA, 25 bp, Artificial Sequence
  Strand of probe
tatcatggccgacaagcagaagaac